(19) United States
(12) Patent Application Publication
(10) Pub. No.: US 2025/0253043 A1
(43) Pub. Date: Aug. 7, 2025

(54) REMOTE HEALTH MONITORING

(71) Applicant: Cirrus Logic International Semiconductor Ltd., Edinburgh (GB)

(72) Inventor: Kevin MOORE, Edinburgh (GB)

(73) Assignee: Cirrus Logic International Semiconductor Ltd., Edinburgh (GB)

(21) Appl. No.: 18/987,310

(22) Filed: Dec. 19, 2024

Related U.S. Application Data
(60) Provisional application No. 63/550,642, filed on Feb. 7, 2024.

(51) Int. Cl. G16H 40/67; G16H 80/00

(52) U.S. Cl. CPC G16H 40/67; G16H 80/00

(57) ABSTRACT

A health monitoring device, comprising: processing circuitry configured to generate a test monitor signal, the test monitor signal configured to imitate a monitor signal of interest; transmission circuitry configured to transmit the test monitor signal over the signal path to a host device, the test monitor signal for determining at the host device an integrity of the signal path for transmission of the monitor signal of interest over the signal path.

### TECHNICAL FIELD

The present disclosure relates to the remote monitoring of health signals.

### BACKGROUND

Remote monitoring systems are increasingly being used for health services provided over telecommunication channels, so-called telehealth services. Such systems allow a physician to monitor vital signs of patients from a remote location. For example, digital stethoscopes can be used for remote auscultation of a subject (e.g. patient), to monitor their heart sound, respiration, and/or digestion.

Audio signals transmitted over a communications channel often undergo filtering which can affect the diagnosis of a subject's condition. Variations in hardware and software used for such transmission, for example from one patient to the next, and from one physician to the next, can lead to additional uncertainty in a received signal. In addition to adverse channel conditions, external noise sources proximate the monitoring device can detrimentally affect signals received by the physician.

### SUMMARY

According to an aspect of the disclosure, there is provided a health monitoring device, comprising: processing circuitry configured to generate a test monitor signal, the test monitor signal configured to imitate a monitor signal of interest; transmission circuitry configured to transmit the test monitor signal over the signal path to a host device, the test monitor signal for verifying at the host device an integrity of the signal path for transmission of the monitor signal of interest over the signal path.

The transmission circuitry may be configured to: transmit verification data to the host device, the verification data defining content of test monitor signal for verifying the integrity of the signal path at the host device. The verification data may comprise an audio file representing the synthesised monitor signal.

The health monitoring device may further comprise a first transducer configured to generate the monitor signal of interest.

The health monitoring device may further comprise an output audio transducer located proximate the first transducer. The processing circuitry may be configured to output the test monitor signal via the output audio transducer, the signal path comprising the output audio transducer and the first transducer.

The health monitoring device may further comprise: a second transducer configured to generate the genuine monitor signal in combination with the first transducer.

The second transducer may be located external to the health monitor device, the second transducer configured to remove noise from the genuine monitor signal.

The test monitor signal may comprise a recording of a representative sound of interest. Sounds of interest comprise one or more of: a heart sound; a digestive sound; a respiratory sound. The test monitor signal may be at least partially synthesized. The test monitor signal may comprise one or more of a sweep of frequencies, a sweep of pitches, and a plurality of tones. The test monitor signal may comprise frequency components outside of a frequency range audible to a human.

The processing circuitry may be configured to combine the test monitor signal with the monitor signal of interest to obtain a combined signal, the transmission circuitry may be configured to transmit the combined signal to the host device over the signal path.

The processing circuitry may be configured to generate the test monitor signal and process the monitor signal of interest simultaneously.

The health monitoring device may further comprise memory, the processing circuitry configured to store the test monitor signal in the memory.

The processing circuitry may be configured to generate the test monitor signal based on a mode of operation of the health monitoring device.

The processing circuitry may be configured to receive an indication of a mode of operation of the health monitoring device from the host device.

The transmission circuitry may be configured to transmit the mode of operation to the host device. The mode of operation may comprise an indication of one or more frequency bands of the monitor signal of interest. The mode of operation may comprise one or more of: a cardiac mode; a respiratory mode; and a digestive mode.

The health monitoring device may comprise a telehealth monitoring device. The health monitoring device may comprise a digital stethoscope.

The health monitoring device may further comprising one or more of the following: a temperature sensor; a photoplethysmography (PPG) sensor; an electrocardiogramaensor; and an accelerometer.

The processing circuitry may be configured to: receive, from the host device, an integrity flag indicating the integrity of the signal path; and in response to receipt of the integrity flag, transmitting, using the transmission circuitry, the monitor signal of interest to the host device over the signal path.

The health monitoring device may comprises one of a wearable device, an analyte monitoring device, an analyte sensing device, a mobile computing device, a laptop computer, a tablet computer, a games console, a remote control device, a home automation controller or a domestic appliance, a toy, a robot, an audio player, a video player, or a mobile telephone, and a smartphone.

According to another aspect of the disclosure, there is provided a health monitoring system, comprising: the health monitoring device of any one of the preceding claims; and the host device communicatively coupled to the health monitoring device, the host device comprising circuitry in the signal path.

The signal path may comprises a voice over internet protocol, VOIP, channel.

The host device may comprise a transducer for playback of the received test monitor signal to a user of the host device.

The health monitoring device and the host device may be substantially identical.

The host device may be configured to determine the integrity of the signal path.

Determining of the integrity of the signal path may comprise providing the received test monitor signal to trained neural network.

According to another aspect of the disclosure, there is provided a host device, comprising: processing circuitry; and a memory comprising instructions which, when execute by the processing circuitry, cause the remote health monitoring device to perform the steps of: receiving, over a signal path, a test monitor signal from a health monitoring device; determine an integrity of the signal path based on the received test monitor signal; receive a monitor signal of interest over the signal path; and process the received monitor signal in dependence on the integrity of the signal path.

According to another aspect of the disclosure, there is provided a method of determining an integrity of a signal path for transmission of a monitor signal, the method comprising: at a health monitoring device: generating a test monitor signal, the test monitor signal configured to imitate the monitor signal; transmitting the test monitor signal over the signal path to a host device, the test monitor signal for determining at the host device an integrity of the signal path for transmission of the monitor signal of interest over the signal path.

The method may further comprise, at the host device, determining the integrity of the signal path for transmission of the monitor signal of interest.

The method may further comprise, at the health monitoring device, in response to receive of an indication from the host device of the integrity of the signal path, transmitting the monitor signal to the host device over the signal path.

Throughout this specification the word “comprises”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the present disclosure will now be described by way of non-limiting examples with reference to the drawings, in which:

FIG. 1 is a schematic diagram of a health monitoring device in situ with a subject of interest;

FIG. 2 a schematic diagram of the health monitoring device shown in FIG. 1;

FIG. 3 is a system diagram of an example implementation of the health monitoring device shown in FIGS. 1 and 2;

FIG. 4 is a block diagram of an example signal chain;

FIG. 5 is a flow diagram of an example process implemented by a local health monitoring device; and

FIG. 6 is a flow diagram of an example process implemented by a remote health monitoring device.

### DESCRIPTION OF EMBODIMENTS

Embodiments of the present disclosure relate to improvements in remote health monitoring. Specifically, various methods are described for the verification of the integrity of a signal path between a health monitoring device and a remote host device.

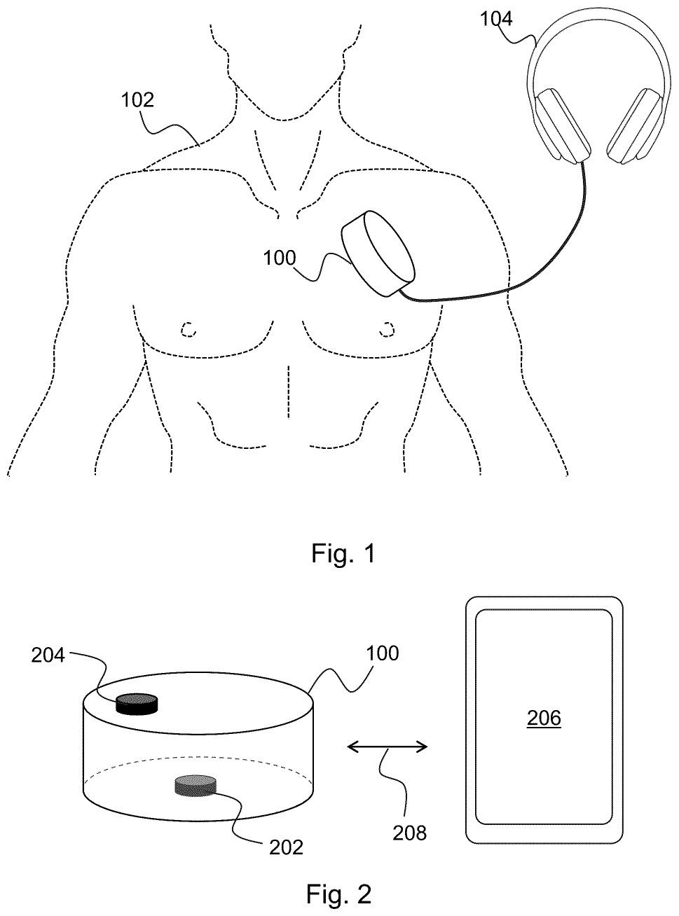

FIGS. 1 and 2 schematically illustrate a health monitoring device 100 according to embodiments of the present disclosure. The health monitoring device 100 is shown in-situ in contact with a subject 102 to be monitored. In this example the health monitoring device 100 is a digital stethoscope. It will be appreciated, however, that embodiments of the present disclosure are not so limited. Optionally, the health monitoring device 100 may be coupled to a local headset 104 which may be used by the subject 102 or a third party local to the headset 104, such as a nurse.

Referring to FIG. 2, the health monitoring device 100 may comprise a first transducer 202 configured to transduce sounds from the subject to generate a monitor signal of interest. The first transducer 202 may be a drum microphone for application to the subject 102. Optionally, the health monitoring device 100 may further comprise a second transducer 204 for use in active noise cancellation (ANC) as will be described in more detail below. The second transducer 204 may be a microphone. The second transducer 204 may be located at a distal end of the health monitoring device 100 distal from the subject 102 in use. The health monitoring device 100 is configured to communicate with a host device 206 over a wired or wireless link 208. For example, the host device may be a mobile device (e.g. smartphone) or a computer.

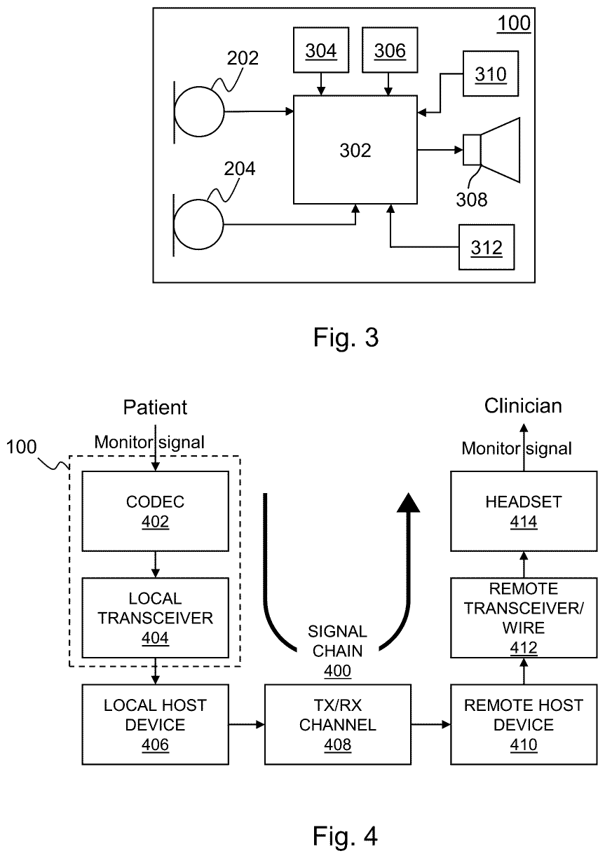

FIG. 3 is a block diagram of the health monitoring device 100 showing the various constituent components. As noted above, the health monitoring device 100 comprises the first transducer 202 and optionally the second transducer 204. In variations of that shown, the second transducer 204 may be omitted.

A processor 302 of the health monitoring device 100 is configured to receive signals from the transducers 202, 204 and process the signals to generate a monitor signal of interest. In addition, the processor 302 may be configured to generate one or more test monitor signals which mimic a given signal of interest, as will be described in more detail below.

The processor 302 may be implemented as a digital signal processor (DSP) or the like. The processor 302 may implement one or more audio codecs, such as the CS47L35 codec from Cirrus Logic, Inc.

The health monitoring device 100 further comprises a memory 304, which may be provided as a single component or as multiple components. The memory 304 is provided for storing data and/or program instructions which may be implemented or executed by the signal processor 302.

The health monitoring device 100 may further comprise a transceiver 306, which is provided for allowing the health monitoring device 100 to communicate (wired or wirelessly) with external devices, such as the host device 206 shown in FIG. 2, to which the health monitoring device 100 is coupled. For example, the health monitoring device 100 may be connected to a network via the transceiver 306 and configured to transmit audio data received at or generate by the health monitoring device 100 to the cloud, a remote server for further processing and/or to a remote device, such as the host device 206. Such audio data may comprise one or both of monitor signals obtained from the first and/or second transducers 202, 204, or test monitor signal generated by the signal processor 302. Communications between the health monitoring device 100 and external device(s) may comprise wired communications where suitable wires are provided.

The transceiver 306 may further comprise an interface (not shown) for coupling to a local headset, such as the headset 104 shown in FIG. 1. The interface may comprise an audio connector or jack for coupling with an input or output health monitoring device, such as the headset 104. The processor 302 may be configured to monitor the interface to detect coupling of an input or output health monitoring device. Additionally, or alternatively, the processor 302 may be configured to monitor if the external component comprises an input audio transducer, such as voice microphone and/or and output audio transducer, such as a speaker. On detection of the presence of any such input and/or output transducers, the processor 302 (or transceiver 306) may be configured switch the health monitoring device 100 between operating modes, such as being configured to receive audio from the connected input/output transducer instead of the first and/or second transducer 202, 204 integral to the device 100. Additionally, or alternatively, a voice signal from a headset voice microphone of the headset 104 may be processed by the processor 302 instead or in addition to a signal from the second transducer 204. For example, audio signals may be output via the jack for feedback to the local operator of the device 100 (e.g. the subject 102 or a clinician local to the subject 102).

Optionally, the health monitoring device 100 may comprise a loudspeaker 308. The processor 302 may be configured to output, via the loudspeaker 308, audio to the user of the device 100. Such audio may comprise instructions, or notifications to the user and/or the monitored signals obtained from the first and/or second transducers 202, 204.

Optionally, the health monitoring device 100 may comprise one or more additional sensors 310. Such additional sensors 310 may comprise one or more of a temperature sensor, a photoplethysmography (PPG) sensor, an electrocardiogra sensor (ECG), and an accelerometer (or gyroscope or inertial measurement unit).

The health monitoring device 100 may be powered by a battery 312 and may comprise other sensors (not shown). It will be appreciated that methods described herein may be implemented on the health monitoring device 100 or on a host device (e.g. the host device 206) to which the health monitoring device 100 is connected, or in the cloud (e.g. on a remote server), or a combination of all three.

FIG. 4 is a block diagram of an example signal chain between a subject or use of the health monitoring device 100 and a clinician or user located at a remote location monitoring signals obtained using the health monitoring device 100. The signal chain is illustrated to show how various components of the signal chain contribute to channel effects on the received monitor signal.

From the perspective of a subject using the health monitoring device 100, the signal chain may comprise a local codec 402 and a local transceiver 404 (implemented as part of the health monitoring device 100. The signal chain 400 may optionally further comprise a local host device 406 configured to process and/or relay audio signals received, processed and transmitted via the transceiver 404. The local host device 406 may be a smartphone or personal computer owned and/or operated by the subject, by a clinician local to the subject, or another third party. Knowledge of specific processing performed by the local host device 406 may not be known to the health monitoring device 100 and/or any device to which signals from the local host device 406 are transmitted.

The signal chain 400 further comprises a transmission channel 408 for transmission of audio signals from the health monitoring device 100 local to the subject 102. Data may be transmitted over the transmission channel 408 using an internet protocol (IP), such as a voice over IP (VOIP) channel. The transmission channel may comprise software, such as a telehealth communications platform or an audio and/or video conferencing suite, such as Zoom (®), Teams (®), or the like. As such, the transmission channel 408 may be affected by noise and/or channel effects associated with the transmission channel 408 and any hardware and/or software used to process transmitted signals. Such effects may lead to uncertainty as to the quality and or integrity of signals travelling through the signal chain 400.

Signals transmitted over the transmission channel 408 may be provided to a remove host device 410. The remote host device 410 may be a smartphone or a personal computer. Like the local host device 406, the remote host device 410 may be owned and/or operated by a remote clinician, or another third party. As such, knowledge of specific processing performed by hardware of the remote host device 410 may not be known to the health monitoring device 100 and/or any device to which signals from the remote host device 410 are transmitted.

Signals received at the remote host device 410 may be transmitted to a headset 414 via a remote transceiver 412, such as a Bluetooth transceiver. Where the headset 414 is a wired headset, a wire may replace the remote transceiver 412 in the signal chain 400.

A monitor signal derived from the first and/or second transducers 202, 204 of the device 100 is processed by codec 402, which may optionally be implemented by the processor 302. The encoded monitor signal is then transmitted by the local transceiver 404, optionally, via the local host device 406 through the transmission channel 408 to a remote host device 410. The received monitor signal is then output by to the headset 414 via the transceiver 412 or wire such that a clinician can listen to the monitored signal representing a health condition of the subject.

It will be appreciated that at each of these stages in the signal chain 400 noise and/or channel compensation may be applied. As is explained above, such noise and/or channel compensation may not be under the control or knowledge of the health monitoring device 100, the local host device 406, and/or the remote host device 410. Such uncertainty in the quality and/or effects applied to the monitor signal output at the headset 414 to the clinician may lead to uncertainty in the integrity of the signal received.

Embodiments of the present disclosure aim to address or at least ameliorate these challenges by generating a test monitor signal at the health monitoring device 100 which is transmitted via the transmission channel 408 to the remote host device 410. By generating and transmitting a test monitor signal over the signal chain 400, properties of the signal chain, such as its integrity, may be ascertained. In doing so, a measure of the viability of the signal chain 400 for transmission of subsequent genuine monitor signals obtained by the device 100, for example from the subject 102, may be obtained to provide clinicians or clinical processing equipment with the confidence that received monitor signals received by the remote host device 410 are a genuine reproduction of signals obtained from the subject 102. Additionally, information regarding properties of the channel obtained from a received test monitor signal may be used on the receiving side of the signal chain 400 to process the received signal to remove unwanted channel effects, so as to reproduce a received monitor signal in a form suitable for interpretation or diagnosis by a clinician or diagnostic processor.

The test monitor signal may be injected at various positions in the signal chain 400. By introducing the test monitor signal early in the signal chain 400, the test monitor signal may be subject to the same suite of channel effects to which a genuine monitor signal may be subject. For example, the processor 302 may be configured to output the test monitor signal at the speaker 308 of the health device 100, and the test monitor signal may then be picked up by the first and/or second transducers 202, 204 and processed and transmitted by the processor 302 and transceiver 306/404 to the remote host device 410. In another example, the codec 402 implemented by the processor 302 may generate the test monitor signal to be transmitted through the signal chain 400. By injecting the test monitor signal at the codec 402, the effect of ambient noise proximate the first and/or second transducers 202, 204 may be substantially ameliorated. In another example, the local transceiver 404 may be configured to generate and inject a test monitor signal directly into the signal chain 400 for transmission to the remote host device 410, bypassing the codec 402.

The test monitor signal may comprise one or more frequencies, pitches or tones. Such frequencies, pitches or tones may be constant and/or swept. The test monitor signal may be configured to mimic or synthesize a signal of interest. For example, as noted above, the health monitoring device 100 may be configured to monitor any conceivable audible heath condition, including cardiac, respiratory, and digestive sounds. As such, the test monitor signal may comprise components at one or more frequencies of interest. Such frequencies of interest may be at frequencies which would be expected in a genuine monitor signal of interest. For example, where the monitor signal of interest comprises heart sounds, the test monitor signal may comprise components at frequencies of heart sounds. More specifically, if the monitor signal of interest is a heart sound comprising a cardiac murmur, the test monitor signal may comprise components at frequencies associated with a heart murmur. In some embodiments, the test monitor signal may comprise a recording of a monitor signal of interest. For example, if the monitor signal of interest is a heart murmur, the test monitor signal may comprise a recording of a heart murmur, which may be injected into the signal chain 400. Additionally or alternatively, the test monitor signal may be at least partially synthesized to mimic the monitor signal of interest or to comprise components characteristic of the monitor signal of interest.

In any case, the test monitor signal may be generated based on a mode of operation of the health monitoring device 100. Modes of operation may pertain to audio signals of interest. For example, the health monitoring device 100 may be operable in a cardiac mode, a respiratory mode, and a digestive mode. In the cardiac mode, the test monitor signal may be generated to mimic cardiac sounds. In the respiratory mode, the test monitor signal may be generated to mimic respiratory sounds. In the digestive mode, the test monitor signal may be generated to mimic digestive sounds.

With knowledge of the test monitor signal being injected into the signal chain 400 hardware and/or software on the clinician side may be used to determine one or more characteristics, such as a transfer function, of the channel. Such characteristics may be used to a) determine an integrity of the channel, and/or b) determine processing to be applied to subsequent signals received over the channel to remove such characteristics.

To aid in processing of the test monitor signal transmitted over the signal chain 400 to the remote host device 410, verification data may also be transmitted over the signal chain 400 to the remote host device 410. Verification data may comprise digital information pertaining to the test monitor signal. For example, the digital information may comprise a mode of operation of the health monitoring device 100 (e.g. cardiac, respiratory, digestive etc.) Additionally, or alternatively, the verification data may digitally describe the test monitor signal as generated before injection into the signal chain 400. For example, the verification data may comprise a digital audio file representing the test monitor signal. The verification data may be used by the remote host device 410 to ascertain the integrity of the signal chain 400. For example, the remote host device 410 may be configured to compare the digital representation of the test monitor signal with the actual test monitor signal transmitted over the signal chain 400. Based on such comparison, the integrity of the channel 408 may be obtained. Additionally, or alternatively, the remote host device 410 may be configured to determine compensation (e.g. filters) to be applied to a subsequently received genuine monitor signal to compensate for effects of the signal chain 400.

The test monitor signal may be injected periodically or continuously into the signal chain 400. For example, the test monitor signal may comprise inaudible components (e.g. above 20 kHz or below 20 Hz), such that the test monitor signal is inaudible to the human ear. The test monitor signal may be combined with the genuine monitor signal and transmitted over the signal chain 400 in combination. This may be necessary where the test monitor signal is injected continuously into the signal chain 400.

The health monitoring device 100 may be configured to transmit, via the transceiver 308, one or more additional signals to the remote host device 410. Such additional signals may be obtained using the one or more additional sensors 310 provided with the health monitoring device 100. Such additional signals may comprise one or more of a temperature signal representing an ambient temperature at the health monitoring device and/or a temperature of the subject 102; a PPG signal representing changes in blood volume in the peripheral circulation of the subject 102; and an ECG signal from the subject 102. Such additional signals may be combined with or sent separately to genuine monitor signals transmitted over the signal chain 400.

As noted above, the health monitoring device 100 is configured to transmit, over the signal chain 400, test and genuine monitor signals of interest. Transmission of genuine monitor signals of interest may be dependent on an integrity of the signal chain 400 as ascertained, for example, by the remote host device 410. For example, on verifying that the signal chain has the characteristics necessary for successful transmission of a genuine monitor signal, the remote host device 410 may be configured to transmit an integrity flag or similar communication to the health monitoring device 100. On receipt of that communication, the health monitoring device 100 may then be configured to transmit a genuine monitor signal to the remote host device 410. Alternatively, the health monitoring device 100 may transmit a genuine monitor signal regardless of the integrity of the channel. In which case, the remote host device 410 may be configured to discard received genuine monitor signals if it determines that the integrity of the signal chain 400 falls below a threshold integrity.

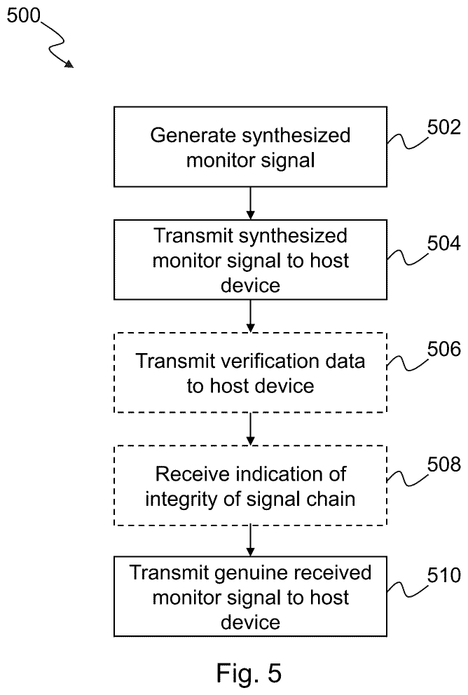

FIG. 5 is a flow diagram of an example process 500 implemented by the health monitoring device 100 described herein.

At step 502, the health monitoring device 100 is configured to generate a test monitor signal. The test monitor signal may be generated using any of the techniques described above. For example, the test monitor signal may be generated to mimic a genuine monitor signal of interest. For example, the test monitor signal may be generated based on a mode of operation of the health monitoring device 100.

At step 504, the health monitoring device 100 is configured to transmit, over the transmission channel 408 and to the remote host device 410, the test monitor signal.

Optionally, at step 506, the health monitoring device 100 is configured to transmit verification data to the remote host device 410 over the transmission channel 408. Such verification data may comprise a digital representation of test monitor signal, an indication of the mode of operation of the health monitoring device 100, and/or an indication of any effects applied by the health monitoring device to the test monitor signal before transmission over the transmission channel 408.

Optionally, at step 508, the health monitoring device 100 may receive an indication of the integrity or quality of the signal chain 400 (e.g. the transmission channel 408). The indication of integrity may be received from the remote host device 410 or another device.

At step 510, the health monitoring device 100 transmits a genuine received monitor signal to the remote host device 410 over the transmission channel 408. Transmission of the genuine monitor signal may be performed in dependence on the indication of integrity, if received. Alternatively, the genuine monitor signal may be transmitted regardless of any indication of integrity (or of step 508 is omitted altogether).

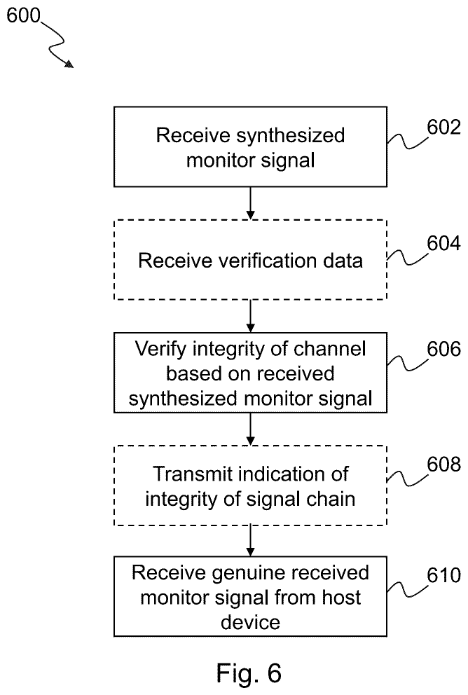

FIG. 6 is a flow diagram of an example process 600 which may be implemented by the remote host device 410 described herein.

At step 602, the remote host device 410 may receive a test monitor signal from the health monitoring device 100 over the transmission channel 408.

Optionally, at step 604, the remote host device 410 may also receive verification data from the health monitoring device 100. The verification data may comprise information pertaining to the test monitor signal.

At step 606, the remote host device 410 may verify an integrity of the channel based on the received test monitor signal, and optionally based on the verification data, if received.

Optionally, at step 608, the remote host device 410 may be configured to transmit to the health monitoring device 100 an indication of the integrity of the signal chain 400 or transmission channel 408.

At step 610, the remote host device 410 is configured to receive a genuine monitor signal of interest, which may be processed or transmitted to the headset 414 for diagnosis by a clinician.

The skilled person will recognise that some aspects of the above-described apparatus and methods may be embodied as processor control code, for example on a non-volatile carrier medium such as a disk, CD- or DVD-ROM, programmed memory such as read only memory (Firmware), or on a data carrier such as an optical or electrical signal carrier. For many applications embodiments of the invention will be implemented on a DSP (Digital Signal Processor), ASIC (Application Specific Integrated Circuit), FPGA (Field Programmable Gate Array), or system-on-chip (SoC). Thus, the code may comprise conventional program code or microcode or, for example code for setting up or controlling an ASIC or FPGA. The code may also comprise code for dynamically configuring re-configurable apparatus such as re-programmable logic gate arrays. Similarly, the code may comprise code for a hardware description language such as Verilog™ or VHDL (Very high-speed integrated circuit Hardware Description Language). As the skilled person will appreciate, the code may be distributed between a plurality of coupled components in communication with one another. Where appropriate, the embodiments may also be implemented using code running on a field-(re)programmable analogue array or similar device in order to configure analogue hardware.

Note that as used herein the term module shall be used to refer to a functional unit or block which may be implemented at least partly by dedicated hardware components such as custom defined circuitry and/or at least partly be implemented by one or more software processors or appropriate code running on a suitable general-purpose processor or the like. A module may itself comprise other modules or functional units. A module may be provided by multiple components or sub-modules which need not be co-located and could be provided on different integrated circuits and/or running on different processors.

Embodiments may be implemented in a host device, especially a portable and/or battery powered host device such as a mobile computing device for example a laptop or tablet computer, a games console, a remote control device, a home automation controller or a domestic appliance including a domestic temperature or lighting control system, a toy, a machine such as a robot, an audio player, a video player, or a mobile telephone for example a smartphone.

As used herein, when two or more elements are referred to as “coupled” to one another, such term indicates that such two or more elements are in electronic communication or mechanical communication, as applicable, whether connected indirectly or directly, with or without intervening elements.

This disclosure encompasses all changes, substitutions, variations, alterations, and modifications to the example embodiments herein that a person having ordinary skill in the art would comprehend. Similarly, where appropriate, the appended claims encompass all changes, substitutions, variations, alterations, and modifications to the example embodiments herein that a person having ordinary skill in the art would comprehend. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative. Accordingly, modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

Although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described above.

Unless otherwise specifically noted, articles depicted in the drawings are not necessarily drawn to scale.

All examples and conditional language recited herein are intended for pedagogical objects to aid the reader in understanding the disclosure and the concepts contributed by the inventor to furthering the art and are construed as being without limitation to such specifically recited examples and conditions. Although embodiments of the present disclosure have been described in detail, it should be understood that various changes, substitutions, and alterations could be made hereto without departing from the spirit and scope of the disclosure.

Although specific advantages have been enumerated above, various embodiments may include some, none, or all of the enumerated advantages. Additionally, other technical advantages may become readily apparent to one of ordinary skill in the art after review of the foregoing figures and description.

It should be noted that the above-mentioned embodiments illustrate rather than limit the invention, and that those skilled in the art will be able to design many alternative embodiments without departing from the scope of the appended claims. The word “comprising” does not exclude the presence of elements or steps other than those listed in a claim, “a” or “an” does not exclude a plurality, and a single feature or other unit may fulfil the functions of several units recited in the claims. Any reference numerals or labels in the claims shall not be construed so as to limit their scope.

1. A health monitoring device, comprising:
processing circuitry configured to generate a test monitor signal, the test monitor signal configured to imitate a monitor signal of interest;
transmission circuitry configured to transmit the test monitor signal over the signal path to a host device, the test monitor signal for verifying at the host device an integrity of the signal path for transmission of the monitor signal of interest over the signal path.

2. The health monitoring device of claim 1, wherein the transmission circuitry is configured to:
transmit verification data to the host device, the verification data defining content of test monitor signal for verifying the integrity of the signal path at the host device.

3. The health monitoring device of claim 2, wherein the verification data comprises an audio file representing the synthesised monitor signal.

4. The health monitoring device of claim 1, further comprising:
a first transducer configured to generate the monitor signal of interest.

5. The health monitoring device of claim 4, further comprising an output audio transducer located proximate the first transducer, the processing circuitry configured to output the test monitor signal via the output audio transducer, the signal path comprising the output audio transducer and the first transducer.

6. The health monitoring device of claim 4, further comprising:
a second transducer configured to generate the genuine monitor signal in combination with the first transducer, wherein the second transducer is located external to the health monitor device, the second transducer configured to remove noise from the genuine monitor signal.

7.-9. (canceled)

10. The health monitoring device of claim 1, wherein the test monitor signal comprises one or more of a sweep of frequencies, a sweep of pitches, and a plurality of tones.

11. The health monitoring device of claim 1, wherein the test monitor signal comprises frequency components outside of a frequency range audible to a human.

12. The health monitoring device of claim 11, wherein the processing circuitry is configured to combine the test monitor signal with the monitor signal of interest to obtain a combined signal, the transmission circuitry configured to transmit the combined signal to the host device over the signal path.

13. The health monitoring device of claim 11, wherein the processing circuitry is configured to generate the test monitor signal and process the monitor signal of interest simultaneously.

14. The health monitoring device of claim 1, wherein the test monitor signal comprises one or more recordings of sounds of interest, wherein sounds of interest comprise one or more of:
a heart sound;
a digestive sound; and
a respiratory sound.

15.-16. (canceled)

17. The health monitoring device of claim 1, wherein the processing circuitry is configured to:
generate the test monitor signal based on a mode of operation of the health monitoring device.

18. The health monitoring device of claim 17, wherein the processing circuitry is configured to:
receive an indication of the mode of operation of the health monitoring device from the host device.

19. (canceled)

20. The health monitoring device of claim 17, wherein the mode of operation comprises an indication of one or more frequency bands of the monitor signal of interest.

21. The health monitoring device of claim 17, wherein the mode of operation comprises one or more of:
a cardiac mode;
a respiratory mode; and
digestive mode.

22.-23. (canceled)

24. The health monitoring device of claim 1, further comprising one or more of the following:
a temperature sensor;
a photoplethysmography (PPG) sensor;
an electrocardiogramansor; and
an accelerometer.

25. The health monitoring device of claim 1, wherein the processing circuitry is configured to:
receive, from the host device, an integrity flag indicating the integrity of the signal path; and
in response to receipt of the integrity flag, transmitting, using the transmission circuitry, the monitor signal of interest to the host device over the signal path.

26. The health monitoring device of claim 1, wherein the health monitoring device comprises one of a telehealth monitoring device, a digital stethoscope, a wearable device, an analyte monitoring device, an analyte sensing device, a mobile computing device, a laptop computer, a tablet computer, a games console, a remote control device, a home automation controller or a domestic appliance, a toy, a robot, an audio player, a video player, or a mobile telephone, and a smartphone.

27. A health monitoring system, comprising:
the health monitoring device of claim 1; and
the host device communicatively coupled to the health monitoring device, the host device comprising circuitry in the signal path.

28. The health monitoring system of claim 27, wherein the signal path comprises a voice over internet protocol, VOIP, channel.

29. The health monitoring system of claim 27, wherein the host device comprises:
a transducer for playback of the received test monitor signal to a user of the host device.

30. The health monitoring system of claim 27, wherein the health monitoring device and the host device are substantially identical.

31. The health monitoring system of claim 27, wherein the host device is configured to determine the integrity of the signal path.

32. The health monitoring system of claim 27, wherein the determining of the integrity of the signal path comprises:
providing the received test monitor signal to trained neural network.

33. A host device, comprising:
processing circuitry; and
a memory comprising instructions which, when execute by the processing circuitry, cause the remote health monitoring device to perform the steps of:
receiving, over a signal path, a test monitor signal from a health monitoring device;
determine an integrity of the signal path based on the received test monitor signal;
receive a monitor signal of interest over the signal path; and
process the received monitor signal in dependence on the integrity of the signal path.

34. A method of determining an integrity of a signal path for transmission of a monitor signal, the method comprising:
at a health monitoring device:
generating a test monitor signal, the test monitor signal configured to imitate the monitor signal;
transmitting the test monitor signal over the signal path to a host device, the test monitor signal for determining at the host device an integrity of the signal path for transmission of the monitor signal of interest over the signal path.

35.-36. (canceled)
